(19) United States
(12) Patent Application Publication
(10) Pub. No.: US 2010/0222852 A1
(43) Pub. Date: Sep. 2, 2010

(54) Apparatus and Method for Decolonizing Microbes on the Surfaces of the Skin and In Body Cavities

(76) Inventors: David B. Vasily, Bethlehem, PA (US); Raymond A. Hartman, Carlsbad, CA (US); Alfred Intintoli, West Chester, PA (US)

Correspondence Address: CHARLES A. WILKINSON, ESQ., 68 EAST BROAD STREET, P. O. BOX 1426, BETHLEHEM, PA, 18016-1426, US

(21) Appl. No.: 12/712,076

(22) Filed: Feb. 24, 2010

Related U.S. Application Data
(60) Provisional application No. 61/154,824, filed on Feb. 24, 2009.
(60) Provisional application No. 61/154,839, filed on Feb. 24, 2009.

(51) Int. Cl. A61N 5/067; A61N 5/06

(52) U.S. Cl. 607/89; 607/88

(57) ABSTRACT

The invention is directed to an apparatus for decolonizing microbes from skin surfaces and body cavities, in particular the decoloniziation of MRSA from nasal cavities using UVC preferably combined with visible light. The device consists of a lightguide, dispensing tip plus accessories, and a housing with a UV source, optical filtering and light collection means, shutter and timer. An internal or external radiometer provides dosimetry information to the operator. The device has additional utility in killing microbes on skin surfaces and beneath nail beds. The lightguide itself comprises the holder for the dispensing tip for use in cavities and as a holder for surface use. The dispensing tips serve to protect decolonization subjects from cross-contamination and may act to shape the nasal cavity and the light distribution pattern of the emitted UVC and visible radiation.

### REFERENCE TO PREVIOUS APPLICATIONS

This application claims priority from U.S. Provisional Application 61/154,839 filed Feb. 24, 2009 in the names of Raymond A. Hartman and David B. Vasily as well as from U.S. Provisional Application 61/154,824 filed Feb. 24, 2009 in the names of Alfred Intintoli and David B. Vasily.

### 1. FIELD OF INVENTION

The present invention relates generally to the field of UV disinfection systems, infection control and methods therefore. More particularly, the present invention is directed to a method and means for disinfecting the nose and the like as well as other adjunctive uses to which the method and means may be put for medical disinfecting.

### TERMINOLOGY

In this application for patent, the following important terminology should be kept clearly in mind.

MED—Minimum Erythema Dose—This is the lowest dose of UV causing pink skin color with distinct edges.

MBD—Minimum Bactericidal Dose—This is the minimum established dose of UV at a particular wavelength to kill a particular bactericidal species usually established in-vitro.

MCBD—Minimum Clinical Bactericidal Dose—This is the recommended clinical dose to be applied to achieve the killing of a bacteria in-vivo.

IR—Irradiance Ratio—A single number representing the highest irradiance of an incident beam of light on the skin divided by the lowest irradiance contained within the same beam of light incident on the skin.

### 2. BACKGROUND OF THE INVENTION

Electromagnetic radiation in the UVC range (230-280 nm) has long been known to kill microbes. UVC has been used since the 1800s to disinfect water supplies and since 1930 to disinfect microbes from air streams and other surfaces. Recently a significant rise in the spread of bacteria called Methicillin-resistant Staphylococcus aureus (MRSA), usually pronounced “mursa” has been widely reported. MRSA has been termed the “superbug” in the public media, and is responsible for an increasing number of MRSA associated deaths. MRSA infections are primarily associated with other established risk factors, including recent hospitalization or surgery, residence in a long-term care facility, dialysis and indwelling percutaneous medical devices and catheters. Infections caused by MRSA outside the health care setting have been reported worldwide and are of significant growing concern. These community-acquired (CA) MRSA infections differ from hospital-acquired (HA) MRSA in that they affect healthy persons without any previously identified risk factors, and result in the formation of skin abscesses and in rare cases fatal sepsis and necrotizing pneumonia. The ability to confine and contain the spread of MRSA, therefore, is a public health priority.

Many of the earliest outbreaks of community acquired or (CA) MRSA infections of healthy persons without previously identified risk factors have occurred in contact sports, such as American football, wrestling and soccer where skin abrasions, frequently referred to as “turf burns”, which increase the risk for skin and soft tissue infections, or SSTI, particularly with respect to infections caused by Staphylococcus Aureus. Consequently, outbreaks of abscesses dues to MRSA among members of professional and college football teams including those stemming from team to team contact have become a high-profile problem. Studies of such outbreaks have shown that the players relative risk, or RR, of infection is strongly correlated with the amount of contact had between persons or players and the amount of prophycatic means such as treatment of abrasions with antiseptics and general cleanliness. Studies have shown that a substantial proportion of cases of Staphylococcus Aureus appear to originate from colonies in the nasal mucosa. The nasal cavity, and more specifically, the anterior nares have been identified as reservoirs for Staphylococcus Aureus. One study has found that almost 20% of healthy individuals almost always carry a strain of Staphylococcus Aureus with 60% of the population carrying a strain internally, and finally less than 20% almost never carrying a strain of such bacteria. It is assumed that those persons carrying a strain of Staphylococcus Aureus are likely also to pick up Methicillin Resistant Staphylococcus Aureus, or MRSA as well if exposed to it and therefore, it would be well to eliminate such bacteria from the nasal mucosa carriers before contact with susceptible populations, such as ill people in hospitals or even healthy persons, injury, or accident prone populations, such as close contact sports teams and the like.

Often MRSA is spread within the hospital setting itself and thence from the hospital setting to community settings through individuals carrying MRSA in the nasal cavity. One estimate is that 2 billion people worldwide carry a strain of Staphylococcus Aureus or S. Aureus, from its usual golden color in colonies in culture dishes, and of these 53 million people carry MRSA, usually in the nasal cavity. These human carriers may not have MRSA in their bloodstream, and they may show no ill effects, but they are able to and do spread MRSA to more settings. Nasal carriage of S. aureus is a significant factor in developing infection; more than 80% of isolates that cause infection originate from the nose.

One important aim of the invention, therefore, is to provide a means to decolonize MRSA or largely eliminate in the nasal cavity with a device that can be used in the medical as well as community settings. Medical protocols using topical drugs for decolonization of nasal carriage of MRSA exist but are not currently used for purposes of infection control, because of fear of exacerbating the problem. Contemporary medical decolonizing procedures often involve swabbing the inside of the nasal cavity with topical antibiotics. Such procedures are repeated twice daily for 5 days. There is concern in the medical community, however, that the antibiotic swabbing procedure may lead to new and more antibiotic-resistant strains of MRSA, so such swabbing is only resorted to in special cases.

Another important aim of the invention is to treat fungal infections, particularly of the nails of the hand and foot, and to decolonize skin surfaces to prevent surgical site infections. The lightguide handpiece accessories for each application will be different.

The following invention disclosures are known to the present applicants and have been taken into consideration in preparing the present application for patent.

U.S. Pat. No. 4,298,005 issued to M. F. Mutzhas on Nov. 3, 1981, entitled “Radiation Apparatus,” incorporates description of an ultraviolet apparatus based upon an earlier German application directed to a mercury vapor UV lamp including cooling. The aim is to filter out the infrared radiation in order to eliminate ereythema due to infrared rays while still maintaining pigmentation radiation, i.e. “tanning”. The aim is to filter out as much infrared as possible while retaining the rays between 320 and 450 nm. Ultraviolet edge filters made from plate glass, an infrared absorption filter and a blue color (violet glass) filters are used to obtain a spectrum transmission in the far infrared of about 6%.

U.S. Pat. No. 4,558,700 issued to M. Mutzhas on Dec. 17, 1985, entitled “UV Radiation Device for Phototherapy of Dermatoses, Especially Psoriasis,” originated in a West German application for patent. Ultraviolet light between 300 and 330 nm seems to be preferred and it is stated that radiation between 800 and 1400 nm is advantageously reduced and radiation above that preferably completely suppressed. Radiation between 330 and 440 nm, is achieved by the use of UV-permeable greenish-yellow glass. Above these wavelengths, screening of the radiation by layers of water approximately 10 nm thick is practical. Blue-violet or black glass filters can, it is said, be used to suppress wavelengths between 400 and 600 nm. Other filter mediums are mentioned including polychromatic polymethyl methacrylate (PMM), polymethyl chloride and polymethlye texephalate. Several specific examples are provided.

U.S. Pat. No. 4,871,559 issued to J. E. Dunn et al. on Oct. 3, 1989, entitled “Methods for Preservation of Foodstuffs,” which is derived from five (5) previously filed applications and continuation-in-part application filed between November 1983 and November 1986, all ultimately abandoned, is directed to preventing the growth of microorganisms in the surface layers of foodstuffs or in some cases throughout foodstuffs by the use of short pulses of incoherent polychromatic light between 170 and 2600 nanometers. Some emphasis is laid upon ultraviolet radiation as part of the polychromatic light, but the heat effect in the surface layers of food products by longer wavelengths is also emphasized.

U.S. Pat. No. 4,910,942 issued to Dunn et al. on Mar. 27, 1990, entitled “Methods for Aseptic Packaging of Medical Devices,” is a continuing application taking priority from the earlier Dunn et al. application which issued into the Dunn et al. U.S. Pat. No. 4,871,559 to a “Method of Preservation of Foodstuffs.”

U.S. Pat. No. 5,871,522 issued to J. B. Sentilles on Feb. 16, 1999, entitled “Apparatus and Method for Projecting Germicidal Ultraviolet Radiation,” discloses a UVC ray collimator for directing UVC rays directly at an operation site for the inactivation of microorganisms particularly where bone surfaces are exposed during an operation. Sentilles indicates that bones are particularly prone to pick up infections when exposed to the air because of the lack of circulation on their surfaces.

U.S. Pat. No. 6,254,625 issued to C. V. Rosenthal et al. on Jul. 3, 2001, entitled “Hand Sanitizer,” comprising UV radiation tubes arranged for irradiating the hands for sanitizing purposes. An ultraviolet lamp having peek wavelength at 254 nm is used for about six seconds for the inactivation of microorganisms. It is disclosed that these can be germicidal UVC lamps, which can be effective against resistant strains of bacteria and viruses, i.e. MRSA. Ultraviolet rays below 184 nm are first used to cause ionization of the air and creation of ozone for disinfection of the hands, and the ozone can then be reconverted to oxygen by a light source having a wavelength above 300 nm. It is stated that such light should be between about 300 nm in the UVB waveband and 380 nm in the UVA waveband, plus 450 nm in the soret waveband, about 550 nm in the visible waveband and between 660 and 720 nm in the near infrared band (which series of bands, more or less bracket the green wavelengths at above 410 nm. It is stated that suitable polychromatic UVC light at the various wavelengths can be provided. Shielding of the eyes is preferably provided from light in the UVA, UVB and UVC bands.

U.S. Pat. No. 6,071,302 issued to E. L. Sinofsky et al. on Jun. 6, 2000, entitled “Phototherapeutic Apparatus for Wide-Angle Diffusion,” discloses a partially backwardly reflecting end for a fiber optic cable used for phototherapy. The reflecting end is transparent and designed to be somewhat wider than the fiber optic cable upon which it is mounted and includes small pieces of differentially reflective particles or particulates of material internally to reflect various wavelength radiation angularly to the side as well as backwards with respect to the angular direction of the fiber optic cable. Up to a certain point, the more particulates in the liquid, the more scatter to the sides and rear. Various scattering mediums are possible, such as silica, alumina or titania, usually apparently in a silicon base liquid.

U.S. Pat. No. 6,960,201 issued to W. E. Cumbie on Nov. 1, 2005, entitled “Method for the Prevention and Treatment of Skin and Nail Infections,” discloses the use of UVC sometimes combined with other wavelengths for the treatment in particular of infected nails particularly toe nails which are particularly subject to deep seated bacterial, and particularly fungal, infections. Cumbie discloses in considerable detail that UVC has superior microbial elimination effects because it seems to damage ribonucleic acid (RNA) as well as DNA preventing microorganisms from reproducing. This, it is indicated, was known before from the Bolton U.S. Pat. No. 6,129,893. At the same time, it is indicated, UVC has a fairly low penetration power so it has little effect on the skin itself. Cumbie indicates that tests indicate that bacteria can be inactivated or rendered unable to reproduce by an amount of UVC only 3 to 10% of the radiation necessary to kill such organisms. A low-pressure mercury lamp is preferred to provide the UVC light. The Cumbie '201 claims are limited to treating nails with UV light.

U.S. Pat. No. 7,306,620 issued to W. E. Cumbie on Dec. 11, 2007, entitled “Prevention and Treatment of Skin and Nail Infections Using Germicidal Light,” is a continuation-in-part from the earlier Cumbie U.S. Pat. No. 6,960,201. Cumbie adding to the new patent an expanded discussion of pertinent prior art and history of the prior development of UV treatment with new emphasis on treatment of skin diseases in general. Cumbie also added further discussion of the availability of tables predicting the particular wavelengths of light to which different microbes are or might be sensitive. The same emphasis upon the use of UVC was retained plus the use of longer wavelengths to destroy microorganisms by denaturization of proteins rather than the dimerization of pyrimidine was added. The combination of UVC with other sources or radiation including sources at 180 to 1370 nm is mentioned and the inactivation of staphylococcus aureus is mentioned, although MRSA is not specifically alluded to. A radiation dose of 6,600 euro-sec/cm² is indicated to be required to inactivate staphylococcus aureus. Suppliers of UV equipment and dosage charts are liberally mentioned. It is also mentioned that the lower UVB range of 280 to 290 nm is almost as germicidal as UVC and a suggestion is made of possible use even of UVA radiation if the target organisms are stressed by the addition of certain substances to the treatment site. It is stated in column 23 lines 14 to 19 that “while longer wavelengths of light are not considered germicidal by themselves, they can act synergistically with germicidal light to inactivate an organism. In the same column lines 55, it is stated that “the effectiveness of multi-spectrum germicidal light for inactivation of organisms at lower overall doses than UVC alone indicates that other parts of the spectrum have germicidal properties. The exact inactivation mechanism is not known, however, it probably is a combination of several mechanisms that act together to render the cell inactivated or incapable of reproducing. In column 24 lines 4 through 8, it is stated “It is likely that there are certain types of radiation that are more effective than others at inactivating organisms or preventing them from reproducing”. These types of radiation are likely contained in the range of pulsed light at (170 to 2600 nm), but other parts of the spectrum may also be germicidal”. In column 25 Cumbie continues in lines 48 to 53, the suggestion of “use of other light spectrums acting synergistically”. “While the UVC and UVB to a lesser extent, range of light is the most potent germicidally, other parts of the light spectrum may be used to further enhance the effectiveness of treatment”.

U.S. Published Application 2003/0018373 published Jan. 23, 2003 to R. Eckhardt et al., entitled “Method and Apparatus for Sterilizing or Disinfecting a Region on a Patient,” discloses the use of ultraviolet light for disinfecting or sterilizing the surface of a patient's body including catheter entrance orifices or incisions and bandages. It also discloses that various ultraviolet apparatuses or emitting apparatuses can be used including in particular mercury vapor lamps, LEDs and the like. A few seconds exposure is disclosed including longer exposures depending upon the sensitivity of the area being radiated. For example, a heavier radiation may be applied to an area covered by bandages. Optical filter use is broadly disclosed to absorb or block undesired wavelengths. Flashing beams of UV are suggested to limit radiation exposure. Diachronic mirrors are suggested for the same purpose.

U.S. Published Application 2003/0191459 to R. A. Ganz et al., published Oct. 9, 2003, entitled “Apparatus and Method for Debilitating or Killing Microorganisms within the Body,” discloses an apparatus for killing or apoptizing microflora in the alimentary track and particularly Helicobacter pylorie which are known to instigate stomach ulcers. The inventor seems to favor direct insertion of an x-ray source contained in a balloon into the stomach, but uses other means also including ultraviolet light applied directly with a mercury vapor lamp in a balloon, but also directed apparently through a fiber optic tube from an external source.

U.S. Published Application 2005/0256553 to J Strisower, published Nov. 17, 2005, entitled “Method and Apparatus for the Treatment of Respiratory and Other Infections Using Ultraviolet Germicidal Irradiation,” discloses the use of ultraviolet radiation provided through a fiber optic system from an external source. The method of procedure is to insert the fiber optic cable possibly as a part of an intrinsic pulmonary viewing system into the lung via the trachea and into one of the major lobes of the lung. The radiation generator is then turned on, and the fiber is slowly withdrawn, bathing the tissues in ultraviolet light of some specific wavelength. The apparatus can be adapted to or be used in conjunction with a video bronchoscope. It is disclosed that culture samples can be obtained and testing done to see what specific wavelength will be particularly effective against a particular microflora.

U.S. Published Application 2005/0256552 to R. L. White, published Nov. 17, 2005, entitled Toenail Fungus Eradiation” divulges a battery powered light which is strapped upon a digit over the nail to expose the nail to apparently visible light rays over long periods such as when sleeping and the like. The theory is that fungal infections tend to grow best in the dark (not necessarily so, although fungi do not use light for making food) and therefore should be inhibited by being exposed to light of long duration.

U.S. Published Application 2005/0267551 to T. S. Bhullar, published Dec. 1, 2005, entitled “Device for Ultraviolet Radiation Treatment of Body Tissues,” discloses an ultraviolet generating device in which an ultraviolet preferably of 253.7 nm or nominally 254 nanometers is produced in a generator box in which the wavelength is adjusted by a fan blowing on the UV bulb to adjust the temperature and thereby vary the wavelength, although different bulbs are used for different wavelengths. Quartz fiber optic cable is used to transmit the UV light. A halogen bulb is provided in a second casing to provide multichromatic or white light. The two beams are passed through fiber optic cable and combined at a borosilicon trifurcation joint which combines both beams and shines them out of a single fiber optic cable upon the area of the insertion into or upon the body that is being treated such as the interior of a blood vessel, the interior of the mouth or the like. A four fiber optic cable extends from the trifurcation joint to an eyepiece.

U.S. Published Application 2006/0167531 to M. Gertner et al., published Jul. 27, 2006, entitled “Optical Therapies and Devices,” discloses broadly the use of ultraviolet light for the treatment of many diseased conditions from atopic psoriasis to lung and heart diseases and including rhinitic sinusitis. The use of optical filters are mentioned, but not detailed. Spectral output conditioners are mentioned, but not detailed. Page 12 contains the principal discussion of filters used and states that heat control may become particularly important where a light-producing element is located near the structure to be treated. This patent document discloses use for treating MRSA in the nose, but does not include the particular irradiating tip included in a later continuation-in-part application.

U.S. Published Application 2006/0173515 to W. E Cumbie, published Aug. 3, 2006, entitled “Alteration of the Skin and Nail for the Prevention and Treatment of Skin and Nail Infections” originally filed Jul. 21, 2005 and based upon Provisional Application 60/649,316, filed Feb. 2, 2005 is directed to the treatment of skin and nails by UVC. This application appears to be in a separate line of applications from the other Cumbie applications in that it suggests not that UVC inactivates microorganisms that infect nails by altering such organism's internal chemistry rendering them innocuous by apoptosis, but instead deactivates or renders the keratin of the nails unsuitable for nutrition of microorganisms, possibly by cross-linking the keratin molecules in some unknown manner.

U.S. Published Application 2006/0212098 to C. Demetriou et al., published Sep. 21, 2006, entitled “Method and Apparatus for Treating a Diseased Nail” treats diseased nails by pulses of electromagnetic radiation based upon it would appear the color of the disease causing organism in culture, the idea being to inactivate the organism by excessive heating. Various laser apparatuses are used and a list of suppliers is included. A particular example of treatment by laser at about 595 nm in the orange spectral range is provided.

Two brief published applications entitled “Method of Treating Nail Fungus Onychomycosis” and “Hand-Held Ultraviolet Germicidal System” by T. Davidson published Oct. 26, 2006 and Apr. 13, 2006 respectively suggest ultraviolet treatment of nail fungus by ultraviolet radiation obtained by a penlite-type apparatus.

U.S. Published Application 2006/0212099 to R. H. Riddell, published Sep. 21, 2006, entitled “Optical Skin Germicidal Device and Method,” discloses projecting ultraviolet light through a fiber optic tube into a needle with an optically transparent slit on one side. The needle may be lined up internally with the optical slit in the needle after the needle is inserted close to a diseased structure and nearby tissue irradiated.

U.S. Published Application 2006/0235492 to L. Kemeny et al., published Oct. 19, 2006, entitled “Phototherapeutical Apparatus and Method for the Treatment and Prevention of Diseases of Body Cavities,” which application originated in Hungary via an intermediate PCT application, discloses the treatment of various nasal conditions with ultraviolet light. All or most types of rhinitis are claimed to be aided by ultraviolet light application which is applied through the apparatus, which is indicated to have gone back more than 20 years for treatment of various allergenic and auto-immune skin diseases. It is preferred to pre-treat the area treated with psoralen, but not necessary. “A number of ultraviolet delivery systems” are mentioned and prior patents, paragraph 0052 mentions various UV generators and particularly laser and LEDs, but also multi-wavelength discharge lasers, such as xenon arc lamps and mercury vapor lamps. The use of optical filters and diachronic mirrors is mentioned. It is also mentioned that in some embodiments, the optical guidance system also “special filtering” of the ultraviolet light beam. A handgun-type handgrip is used to direct the light into the nasal cavity. The intensity of radiation is adjusted by first irradiating an un-sunburned portion of the body to determine a so-called minimum photoxicity doses or MPD and MJ/cm³ and/or a minimal erythenol dose or MED.

U.S. Published Application 2007/0219600 to Gertner et al, published Sep. 20, 2007, entitled “Devices and Methods for Targeted Nasal Phototherapy,” is a continuation-in-part application zeroing in specifically on the alleviation in the nasal cavity of MRSA and showing various application methods. The application was not merely a copy of the original application with new material added at the end, but a substantial rewrite of the entire application and shows evidence of a new search having been done. Clinical examples are included in the write-up at the end.

U.S. Published Application 2007/0255266 to W. E. Cumbie, published Oct. 19, 2007, entitled “Method and Device to Inactivate and Kill Cells and Organisms That are Undesirable,” discloses the use particularly of pulsing ultraviolet wavelengths to obtain more penetrating ultraviolet rays to deactivate or kill by apoptising or gene death. This application discussed screening out wavelengths that are not desirable. It also has an extensive discussion of relative penetration of A, B and C ultraviolet radiation indicating that the longer A and B ultraviolet rays are more penetrating, but the shorter C ultraviolet is more germicidal.

U.S. Published Application 2007/0255356 to Rose et al., published Nov. 1, 2007, entitled “Photodisinfection Delivery Devices and Methods,” discloses a kit for treating various bacterial infections including MRSA, in which an applicator section made of various materials and having the property of allowing electromagnetic radiation to spread out is fitted over the end of a wave guide. The applicator section is inserted into the anterior nares of the nose and allows the electromagnetic radiation passing from the end of the wave guide upon the end of which it is fitted to spread out and irradiate the inside of the nose, ear or other orifice in which it is placed subsequent to the application of a photosensitization agent to the body cavity, which is then activated by the proper wavelength, usually ultraviolet, but also other wavelengths with single wavelengths or multiple wavelengths. The sensitizers are of various types, but particularly so-called type 1 and type 2 photosensitizers, the first of which releases a free radical when activated by the proper electromagnetic radiation and the second of which releases single oxygen atoms when activated. The preferred photosensitizer is phenothiazines, such as methylene blue or toluidine blue. The preferred electromagnetic radiation may be from an LED or laser such as the Periowave™ laser light system using wavelength range of 665 nm to 675 nm. Various time-periods of the radiation are mentioned including 15 seconds to 5 minutes at with 30 to 90 seconds at 1 to 25 J/cm². Multiple cycles of light may be applied. It is stated that it is “preferred” that application of light to the site does not cause physiological damage to the host tissues. Positive lab results are cited, including the prophylactic treatment of MRSA in the anterior nares.

U.S. Published Application 2007/0255357 to A. Rose et al. published Nov. 1, 2007, entitled “Nasal Decolonization of Microbes,” directed to the method of sanitizing the anterior nares uses much the same information with additional laboratory data as the above as the co-pending Rose application. It also uses much the same material with additional data regarding laboratory tests.

U.S. Published Application 2008/0119914 to A. Rose et al. published May 22, 2008, entitled “Treatment for Otitus Externa,” uses much the same information as the two earlier Rose applications with additional equipment and lab tests with respect to Otitus Externa.

While there have, therefore, as evidenced by the foregoing documents, been multiple attempts to develop widely applicable apparatus and methods for the application of ultraviolet radiation to desired portions of the human body for the alleviation of various conditions all such previous developments have failed in one regard or another to develop a really practical and safe use of ultraviolet radiation for direct elimination of substantial numbers of resistant organisms, either internally, or on the surface of the body without substantial harm to the bodily cells themselves. In particular, this has been so with respect to the substantial elimination of A. Staphylococcus from the nose and also to a lesser extent of fungal agents from under the finger and toenails as well as some other locations on the body. However, the present inventors have now provided a very practical, effective and safe apparatus and method for accomplishing such aims.

### OBJECTS OF THE INVENTION

It is an object of the present invention, therefore, to provide an apparatus for irradiation of portions of the body to eliminate pathogenic organisms.

It is a further object of the invention to provide an apparatus for the elimination of pathogenic organisms from the human body wherein such organisms are partially shielded from direct radiation by portions of the body itself.

It is a still further object of the invention to provide a radiation apparatus which directs ultraviolet light upon partially protected areas of the body using a hand instrument on the terminus of a flexible connection for ease of application.

It is a still further object of the invention to provide a flexible radiation conducting cable that blocks out those rays from a radiation detector which may have unwished effects.

It is a still further object of the invention to filter out substantially all electromagnetic rays except the ultraviolet light rays which are useful to kill or inactivate pathogenic micro-organisms and a simple range of visible light to serve as an indication of radiation and serve thereby as a warning the apparatus is radiating.

It is a still further object of the invention to use a visible light source that is itself destructive to pathogenic micro-organisms.

It is a still further object of the invention to provide an apparatus that effectively eliminates so-called MRSA from the nasal nares.

It is a still further object of the present invention to provide an apparatus for treating bodily nail beds to eliminate pathogenic fungal organisms.

It is a still further object of the invention to filter out unwanted rays and particularly infra-red rays from a full radiation beam leaving only the rays necessary to kill to inactivate the micro-organisms deemed to be harmful.

It is a still further object of the invention to use a protective and bodily surface molding cover over a tip on a radiation dispensing head associated with a flexible dispensing means which tip will smooth out the folds in the interior nares of the nose to allow an even dose of radiation to be applied within the nasal cavity.

It is, furthermore, a still further object of the present invention to provide a new and improved apparatus and method for decolonizing microbes in body cavities, specifically the decolonization of MRSA in the nasal cavity, and for killing microbes on skin surfaces and under nail beds.

It is another object of the invention to provide a nasal decolonization device for MRSA that has short therapeutic treatment times. In a preferred embodiment, the wavelength of the UVC radiation is between 230 nm and 280 nm and the decolonization of MRSA in a nostril can be accomplished in less than one minute.

It is another object of the invention to use visible light to augment the antimicrobial activity of the UVC. The visible light will additionally provide visual warning of emitted invisible UVC and to provide an aiming beam when the device is used to sterilize surfaces.

It is another object of the invention to standardize the shape of the surface of the anterior nares into a cylinder by means of a UVC transparent sleeve when used for nasal MRSA decolonization. It is yet another object of the invention to provide for a reflective surface being inserted into the nostril to allow for backscattered radiation to irradiate surfaces on a perpendicular bias to the central axis of the nostril cavity.

It is another object of the invention to irradiate the nasal cavity with a controlled beam of light in which the maximum irradiance on any portion of the skin is no more than 3 times the minimum irradiance on any portion of the skin. This irradiance ratio is met by shaping the output of the UVC source through transmission through a lightguide and reflective elements placed inside the nostril.

It is a still further object of the invention to use a convertible tip on the end of a radiation dispensing means so the apparatus can be used alternatively for control of MRSA in the nose and fungal organisms under the nails.

Other objects and advantages of the invention will become evident from a careful review of the following description and appended drawings.

### SUMMARY OF THE INVENTION

The present invention provides an electromagnetic ray apparatus providing radiation in the range of ultraviolet and particularly ultraviolet C range, and method by which pathogenic micro-organisms may be eliminated readily from the nares of the nose and also from under the nails. It is a characteristic of the invention that radiation in two disparate ranges, one in the ultraviolet and one in the visible spectrum are radiated at the same time, the visible wavelengths being also lethal to may microorganisms and thus having either an additive or even a synergistic effect with the ultraviolet radiation and also serving as a visible safety beam evidencing that the apparatus is energized.

### BRIEF DESCRIPTION OF THE DRAWINGS

FIG. 1 shows a top sectional schematic view of the device with major operating components of the base unit shown.

FIG. 2 illustrates sectional views of three types of UVC lamp sources that may be used in the base unit. FIG. 2-1 illustrates a high-pressure short-arc mercury lamp with an integral reflector, FIG. 2-2 illustrates a short-arc xenon flashlamp with integral reflector and lens and FIG. 2-3 illustrates a coherent UV laser source and lens for converging light generated into a lightguide.

FIG. 3 shows the cylindrical configuration for a nasal sleeve in accordance with the invention.

FIG. 4 shows three alternative detailed cross sectional configurations of the radiation dispensing head. FIG. 4-1 is a UVC transmissive sleeve with a conical reflective element in the distal tip. FIG. 4-2 shows the same dispensing tip as FIG. 4-1 with additional sleeving on the tube to act as an attenuator or diffuser that may be used to homogenize sections of the light beam or intensity if desired. FIG. 4-3 illustrates that the reflective element may have different shapes.

FIG. 5 shows the output spectrum from 230 nm to 560 nm of the device built to effect the invention using a high-pressure mercury short-arc lamp and dichroic optical filters.

FIG. 6 Illustrates the major variances of nasal opening encountered in normal human anatomy.

FIG. 7 illustrates a cutaway view of a human nostril. FIG. 7-1 shows the expanding nature of the nares posterior to the nasal opening, and FIG. 7-2 illustrates the vestibular shelf that is shielded by line of sight from the external opening of the nares.

FIG. 8 illustrates the use of the nasal sleeve with the lightguide and dispensing tip. Numeral 8-1 represents the lightguide with the dispensing tip attached, and 3 represents a hollow quartz tube configuration of the nasal sleeve before its insertion into the nasal cavity. FIG. 8-2 shows the dispensing tip and nasal sleeve inserted into the nares.

FIG. 9 illustrates in 3-dimensions the hemispherical output 9-1 of a lambertian emission source with a cylinder 9-2 in the center representing a nostril. The cylinder is shown as segmented into 6 equal sub-cylinders. The sub-cylinder closest to the emission source represents an area proximal to the lamp, and the sub-cylinder that intersects the hemispherical surface represents the most distal portion of the nostril. The emission source is represented by a low-pressure mercury lamp 9-3 with an aperture at the center of the hemisphere and coincident with the entrance of the bottom of the cylinder.

FIG. 10 illustrates a two-dimensional cross sectional view of FIG. 9. The subtended angles shown originate at the center of the lambertian emitter and the arcs subtend the areas defined by the upper and lower boundaries of each sub-cylinder.

FIG. 11 illustrates the subtended angles of radiation from a 29 degree NA lightguide positioned ¾″ from the end of a 1″×⅜″ cylinder. This cylinder represents a cross-sectional visualization of a nostril. The subtended angles shown originate at the center of the lightguide beam and subtend the areas defined by the upper and lower boundaries of each sub-cylinder.

FIG. 12 is a graph of experimental data illustrating the ratios of measured irradiance levels of each sub-cylinder shown in FIG. 11 using the device disclosed herein. The irradiation level found with cylinder 1 serves as the point of reference for the irradiation ratios of succeeding cylinder sections.

FIG. 13 is a photograph of agar plates colonized with MRSA bacteria and treated with light from the device. The area of killing of the MRSA by light irradiation as a function of exposure time is shown on the upper plates. In comparison, the control plate on the bottom of the photo shows no bacterial killing. The experimental conditions are described below in Example 1.

FIG. 14 is a photo of MRSA cultures taken from tests tubes that were irradiated from the top of the tubes for the times shown on the photograph. Nearly complete irradiation of the bacteria occurred as detailed below in Example 2.

### DETAILED DESCRIPTION OF THE PREFERRED EMBODIMENTS

The following detailed description is of the best mode or modes of the invention presently contemplated. Such description is not intended to be understood in a limiting sense, but to be an example of the invention presented solely for illustration thereof, and by reference to which in connection with the following description and the accompanying drawings one skilled in the art may be advised of the advantages and construction of the invention.

Decolonization of nasal MRSA using antibiotics is seldom used in infection control protocols because the widespread use of antibiotics has already lead to an increasing incidence of resistance to mupirocin, the topical antibiotic currently most often used to treat colonized nares. The medical community currently avoids the widespread prophylactic use of antibiotics such as penicillin for the same reason. However, UVC has not been demonstrated to produce similar antibiotic resistance even on a long-term basis, and can be used as an important new tool in infection control. Decolonizing the nasal cavities of potential MRSA carriers at strategic traffic chokepoints in MRSA infected settings has been an unmet healthcare priority. The present invention allows for decolonization of MRSA carriers without the dangers of developing new strains of bacteria with even more antibiotic and other resistance. The device can be used, most importantly, for immediate decolonization of DNA-probe positive MRSA carriers, who are being now frequently identified upon hospital admission, thus reducing the risk of post-op infection and spread to other hospital personnel and patients and as well as the general environment.

Decolonization of MRSA in the nasal cavity is different from phototherapy of the nasal cavity. Phototherapy is designed to change the attributes of the human skin cells being irradiated, whereas the purpose of the present invention is to kill or inactivate the microbes on the surfaces within the body cavity or upon the skin. The purpose of decolonization is not to provide therapy to the skin or therapy to an infected wound. The decolonization of MRSA involves generally only the anterior nares, the two nasal cavities (nostrils) separated by the nasal septum. Since the MRSA colonization depth in the nares is considered to exist less than 1 cm from the nasal opening, the deep nasal cavity probes, endoscopes and visualization equipment described by Bhullar (U.S. Pat. No. 7,201,767), Gertner (U.S. Pat. App. No. 2006/0271024) and Kemeny (U.S. Pat. App. No. 2006/0155349), are not required. The dispensing tip of the present invention easily accesses the targeted area of the anterior nares without need for adjunctive endoscopic visualization techniques.

Prior art UVC devices for nasal decolonization such as shown in U.S. Pat. App. No. 2008/0065175 (Redmond) and U.S. Pat. App. No. 2008/0208297 (Gertner), but such earlier technologies do not address all the problems involved with using UVC in the nasal cavity. Using UVC in the nasal cavity is complicated by irregularly shaped nostril geometry, lack of UVC skin protection of the epithelium of the posterior nares and sinuses that do not have a cornified skin layer, horizontal nasal surfaces that cannot be irradiated from the outside of the nostril opening and difficulty in the achievement of a bactericidal dose of UVC without causing a phototoxic reaction that can endanger the patient. All of these problems are addressed in the current invention.

An important consideration in using UVC for nasal or other body cavity decolonization involves the phototoxic effects of excess UVC on the skin. A balance must be achieved to deliver a minimum bactericidal dose (MBD) sufficient to kill MRSA but simultaneously the dose should not be so high that dangerous phototoxic events occur.

Many prior art devices that irradiate skin ignore the delicate balance of UVC dosing that will achieve bacterial killing yet not induce harmful phototoxic reactions. With the irregular geometry of the nostril or uneven aiming of a light probe it is likely that some areas will receive high doses and other areas will receive lower doses. It is an object of this invention to overcome the problems of prior workers in achieving this dosing balance by standardizing the shape of the anterior nares so that a predictable geometry exists for the surfaces to be treated. Also the device provides accurate UVC dosimetry and the beam shape ensures that the dosing of UVC is sufficient to kill the bacteria without harming the patient.

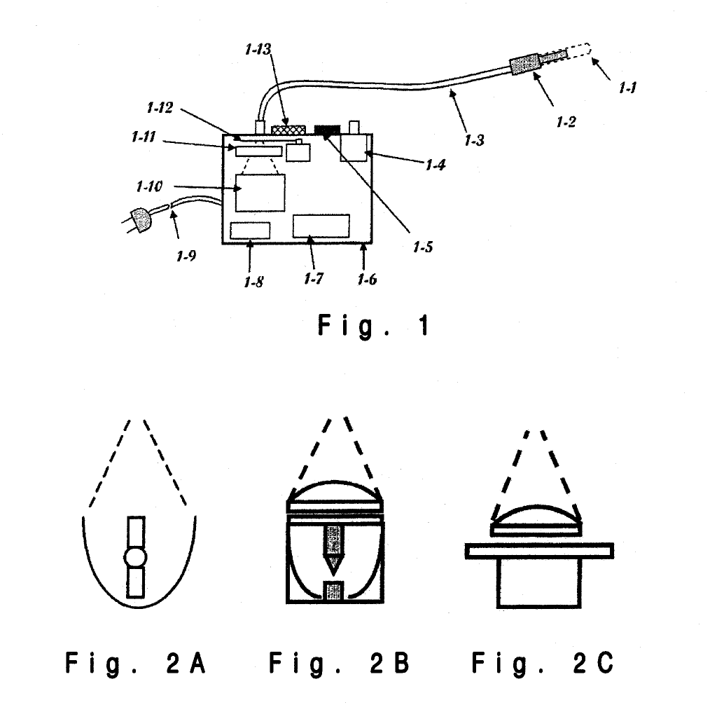

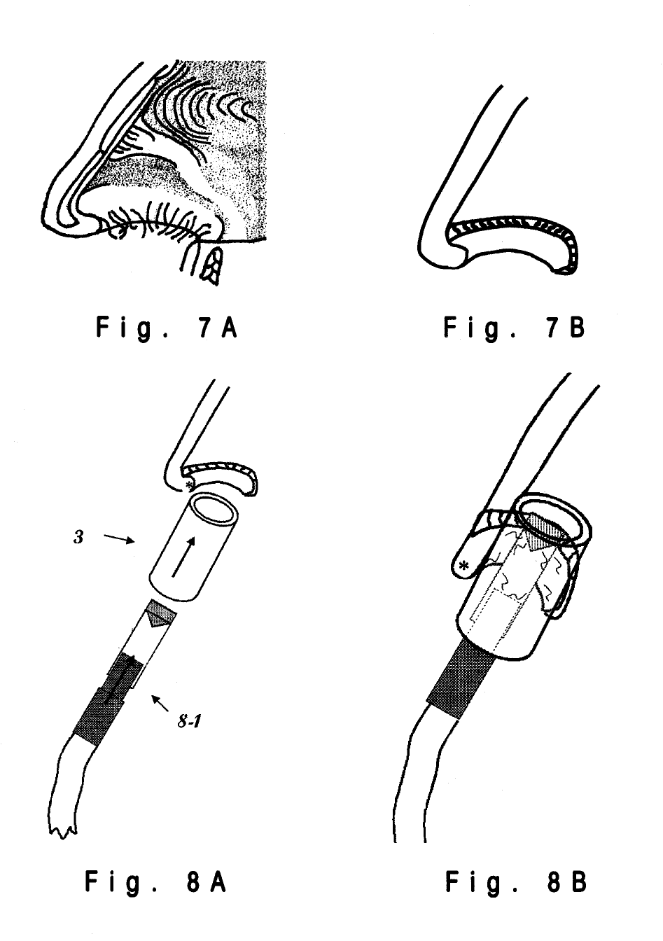

The anterior nares are characterized as having wide anatomical variations in size and shape as discussed in connection with the discussion of FIG. 6. The opening of the nares is rather small and the nasal cavity expands outward in lateral directions past this opening as shown in FIG. 7-1. Horizontal surfaces exist immediately past the nasal opening in an area called the nasal vestibule that is shown in FIG. 7-2. The nasal vestibule is the most anterior section of the nostril and is covered with the same stratified and keratinized skin as normal external skin. This keratin layer protects the underlying proliferating cells from the mutagenic potential of UVC radiation. The nasal vestibule has a shelf-like surface that can harbor bacteria that is shadowed from light entering the nasal opening since they are out of the line of sight. Light entering the opening of the nares because of its straight propagation cannot travel around corners and irradiate the vestibular shelf.

There are LED devices proposed for insertion into the nasal cavity. The light output form these devices cannot travel backward to the vestibular shelf since they have shadowing effects from their packaging bases. There are no omni-directional LED emitters, and at best these LEDs can emit light in a forward hemispherical pattern.

The areas shadowed by the vestibular shelf are most prominent in the frontal tip area known as the infratip lobule. In order for light to reach the top surface of the vestibular shelf in this area the light must enter into the nostril and be reflected backward to the surface of the vestibular shelf. No prior workers have suggested that devices be developed for placing a reflective surface inside of the nostril to provide for radiant energy to be reflected back to the surface of the vestibular shelf.

The shapes of the nasal opening can be described as triangular, oblate, or rhomboidal. The geometry of the anterior nares is uneven and irregular which complicates the homogeneous application of energy to the surface. An inhomogeneous application of energy can lead to underdosing of UVC in some areas thus not killing the bacteria, or overdosing in other areas causing a phototoxic event.

The invention described herein makes the geometrical shape of the anterior nares predictable and provides for flattening the horizontal surfaces past the nasal opening. Flattening of the horizontal surfaces provides for greater exposure of these surfaces to the light from the dispensing tip since the shadowing effect around the nasal opening is reduced. A tight-fitting cylindrical quartz sleeve is inserted into the anterior nares to achieve the predictable geometry. The flexible and expandable nature of the nasal tissue allows for a close fit for the sleeve. The nasal tissue expands and conforms to the cylinder giving a reliable and accessible surface for applying the UVC radiation. Different sized sleeves are used to create a tight fit for different sized nostrils.

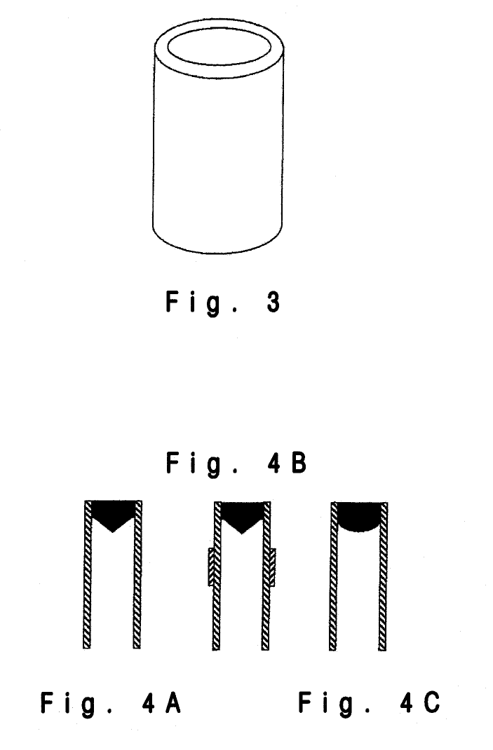

The invention also incorporates a nasal dispensing tip for the lightguide with a reflector element positioned forward of the lightguide in the distal portion of the dispensing tip to reflect radiation both sideways and backwards. This downward radiation provides UVC to any horizontal surfaces that exist just past the nasal opening. The reflector is held in place by a UVC transparent sleeve attached to the end of the lightguide. FIG. 8-1 discussed below illustrates the use of the nasal sleeve and dispensing tip containing the reflective element. The dispensing tip FIG. 4-1 is mounted to the lightguide as shown in FIG. 8-1. The hollow nasal sleeve FIG. 3 is inserted into the nasal opening flaring the nostrils and flattening the vestibular shelf. Then the dispensing tip is inserted into the nasal sleeve and radiation is delivered to the surfaces of the nares.

The reflective element in the dispensing tip also adds a significant protective function to the posterior nares and sinuses. These posterior surfaces are not covered by epithelial tissue that is keratinized and therefore they lack UVC protection for the proliferating cells. Without the blocking action of the reflective tip the UVC could penetrate deeper into the nasal passage and cause significant phototoxic events such as blistering, edema and potential mutagenic action in this unprotected skin. Prior art devices do not prevent UVC from reaching the posterior nares and sinus areas.

Light entering an opening to a cylindrical cavity does not irradiate the side of the cavity evenly, and this irradiance-unevenness is another one of the significant problems of prior devices. This problem emanates from the lambertian light sources incorporated in these devices. From everyday experience, we know that sunlight entering a tunnel or cave only illuminates the sides of the tunnel or cave for a short distance at the entrance. Diffuse light entering a nasal cavity opening also deposits most of the radiant energy along the sides of the initial portion of the nasal cavity. Of course, if the light is shaped, for example into a projecting beam, the depth of penetration into the cavity and evenness of the illumination may be improved. Since excess UVC applied to the skin can cause injury, using a non-directed or unshaped beam entering a nasal cavity can result in an over-application of UVC at the entrance of the nasal cavity and an under-application of UVC in the distal portions of the cavity.

Examples of prior art that result in an uneven application of UVC are illustrated in U.S. Pat. App. No 2008/0065175 (Redmond), which uses UVC generated from low-pressure mercury lamps, and U.S. Pat. App. No 2008/0208297 (Gertner) that uses diffuse light from LEDs. Low-pressure mercury lamps have lambertian radiation patterns, and lambertian patterns cannot achieve the illumination evenness necessary for both procedural efficacy and patient safety. LED light that is diffused along a tubular section also acquires a lambertian irradiation pattern. It will be demonstrated below that a device incorporating a lambertian emitter cannot be used to illuminate the entrance of the nostril and achieve the requisite homogeneity for efficacy and safety.

With the quartz sleeve described herein the nares may be thought of as small cylinders about ⅜″ in diameter and 1 centimeter in depth. It will be geometrically demonstrated that a lambertian emitter such as a low-pressure mercury lamp placed at one end of a cylinder (representing the entrance of the nostril) will apply radiation at a rate much faster in the proximal part of the cylinder than the distal part of the cylinder. As will be further described herein, if sufficient UVC by these prior art devices is placed on the distal part of the nares to kill MRSA, the proximal part of the nares would be severely burned and the safety of the patient would be compromised.

By definition, a lambertian emitter like a low-pressure mercury lamp emits equal amounts of radiation in all directions, and every equal solid angle of radiation emitted from the source contains the same energy of every other equal solid angle. If viewed in two dimensions every degree of included angle has the same energy of every other degree of included angle.

Efficient collection of radiation into a lightguide requires a point source like a short-arc mercury or xenon lamp and a collection means such as an elliptical reflector or a parabolic reflector and converging lens. Additionally the ideal light source for the UVC would contain other wavelengths that can be used to act synergistically with the UVC for destruction of bacteria and fungus. It is known that some visible wavelengths provide antimicrobial activity. Human tissue is highly sensitive to UVC radiation but relatively insensitive to visible radiation. Effectively the addition of certain visible wavelengths lowers the total UVC required to kill microbes, and thus provides additional protection to the skin from the potentially harmful effects of UVC radiation.

In addition to the inherent beam shaping provided by the numerical aperture of the lightguide, additional shaping of the UVC onto the surface of the nares can be accomplished with the dispensing tip. The reflecting element in the dispensing tip reflects back radiation that would normally go into the posterior nares or sinuses. The backward reflection of this radiation provides UVC for bactericidal activity to the off-axis or horizontal surfaces of the vestibular shelf, a surface not accessible directly from the nasal opening.

The addition of visible light is also important because UVC is invisible yet harmful to the human eye. An essential safety feature of ultraviolet devices is to provide warning to the operator, patient and bystanders when radiation is being emitted from a device. In one embodiment, this warning beam is bright enough to cause a bystander to avert their eyes to prevent corneal damage. The brightest wavelength to the human eye is at 555 nm according to the human Photopic Response curve. The atomic emission line of the mercury lamp at 546 nm has nearly the same brightness level and was selected in one embodiment of the device to provide this warning and aiming beam. This wavelength was also selected because green light at a fluence of 8 J/cm2 is known to be effective in killing Trichophyton rubrum, one of the common fungus varieties responsible for human nail fungus infections. In another device embodiment, the visible portion of a xenon flashlamp is contained in the beam and the visual brightness due to the high peak power of this lamp is an effective warning light.

Another value of the short-arc lamp sources is an abundance of visible light that can be employed for antimicrobial activity. Visible light has been found to be effective in killing bacteria including MRSA and the fungus responsible for onychomycosis. This visible light is not available in any significant quantity in low-pressure mercury lamp or monochromatic UVC LEDs disclosed in prior art.

In summary the device disclosed herein overcomes the unevenness of UVC skin application inherent to prior art designs. This is accomplished through light source selection, lightguide transmission and beam shaping, nasal cavity shaping with a UVC transparent sleeve, cavity centering provided by the dispensing tip and optical shaping of the near collimated light by lenses and filters in the base unit and reflective elements in the tip placed into the nasal cavity. The design provides additional microbe killing capacity to complement the UVC by through the use of visible light waves that are antimicrobial but not phototoxic to the skin.

According to one aspect of the invention therefore a UVC apparatus is provided which comprises a base unit having an output port of delivery of UVC radiation within a predetermined spectral range, and optical guide having an input end connected to the output port of the base unit, and a dispensing tip for the lightguide, the base unit including a UVC radiation source, and means for collecting and focusing the UVC into the input end of the lightguide. A quartz cylindrical sleeve is used with the dispensing tip when performing nasal MRSA decolonization. The dispensing tip for nasal MRSA decolonization is comprised of a UVC transparent tube with a reflective element in the distal tip. In one embodiment, the base unit would include a radiometer to measure the output of the lightguide and tip, a shutter and timer or other means of controlling the light input into the lightguide, and a display that would inform the operator as to the UVC output from the lightguide allowing the operator to determine and set specific doses of UVC for the varied intended germicidal purposes.

The device achieves short treatment times by selecting a more energy-dense UVC arc source rather than a more electrically-efficient UVC arc source such as low-pressure mercury lamps, then filtering unwanted radiation from the arc, and transporting the filtered radiation by the highly efficient means of total internal reflection (TIR) through a lightguide. Since lightguides can only efficiently collect and transmit radiation from near-point sources, the UVC lamps used in the system must be mercury short-arc lamps or xenon short-arc flashlamps, both of which are rich in UVC production.

Given such an irradiance ratio the maximum phototoxic response of the patient will be at a 3 MED level, which is an acceptable and safe phototoxic response for all patient populations. A dosing level of 3 MEDs of UVC radiation will provide for erythema without pain and without edema. One MED of UVC is approximately 15 mj/cm2 which is also the 3 log kill dose for MRSA.

It is recognized that the antimicrobial action and control of the phototoxic response will require proper UVC dosimetry. The design provides for UVC dosimetry by an internal radiometer in the preferred embodiment.

Referring to FIG. 1 the base unit of the device consists of a housing 1-6 containing provisions for electrical input 1-9, a UVC light source 1-10 with provision for a associated UVC light source drivers 1-8 (ballast, pulsing electronics), a shutter 1-12 to block the radiation from the source to the lightguide (shown with an attached solenoid activator), a timer 1-13 for controlling the shutter, a display 1-5 to indicate the light output detected by radiometer 1-4. The base unit also provides for low voltage power supply 1-7 to provide suitable power for the components of the base unit.

Lightguide 1-3 may be a UVC liquid lightguide available from Newport Corporation (Irvine, Calif.) or a fused silica fiber bundle. In a preferred embodiment, the lightguide is a UVC liquid lightguide with a 3 mm core. This lightguide is capable of collecting incoming light at a total input angle of 50 degrees, which allows for efficient light collection from an ellipsoidal-reflector short-arc lamp. Alternatively, parabolic reflectors with a focusing lens assembly can be used to collect and focus the light. Another advantage of using liquid lightguides is the assurance that the radiation emitted from the lightguide is non-thermal. UVC liquid lightguides as disclosed by Nath (U.S. Pat. No. 6,418,257) are largely comprised of water. The water in the lightguide absorbs the heat emitted from the lamp source and this assures that the treatment subject will not encounter thermal burning or discomfort.

It is known that a general germicidal action spectrum exists with a peak around 265 nm, and the germicidal activity of mercury lamp radiation at 254 nm is well documented. The device shown schematically in FIG. 1 has filters 1-11 for shaping the spectral output of the UVC source 1-10 to the desired wavelength composition. These filters can be comprised of dichroic-coated fused silica to eliminate unwanted radiation. It is important to avoid UVB radiation in the 290 nm-315 nm range, which may provoke erythema leading to a phototoxic reaction. Radiation in this UVB range has very little antimicrobial activity compared with UVC, yet has very high phototoxic potential.

It is another object of the invention to incorporate accurate dosimetry that allows the user to measure the UVC output from the dispensing tip 1-1 inserted over lightguide end-coupling 1-2 of flexible lightguide 1-3. The radiometer 1-4 can be comprised of a solar-blind UVC photodiode based for example on silicon carbide or gallium nitride. The output of the photodiode is sent to the display 1-5 which can consist of a numeric display or an LED array that would indicate the output irradiance. With the calibration information the operator can set the timer 1-15 that controls the open time of shutter-solenoid 1-12 to deliver the desired UVC dose.

As will be outlined herein, the source of the UVC must be a short-arc mercury lamp as shown with an integral reflector in FIG. 2-1 or a short-arc xenon flashlamp as shown in FIG. 2-2. Only these types of lamps provide the UVC energy density in a point source suitable for efficient collection into a lightguide and also contain visible radiation that is antimicrobial and serves safety functions. A UVC laser with a collimating lens is shown schematically in FIG. 2-3 as a less preferred source of the UVC radiation.

For MRSA nasal decolonization a hollow quartz sleeve FIG. 3 is inserted approximately 10-12 mm into the nostril with enough sleeve projecting outside the nostril to act as a handle for retraction. The sleeve is sized prior to insertion to make a tight fit into the anterior nares and expand and flare the nostril being treated. It will be evident to those skilled in the art that a reflective element can be incorporated into the nasal sleeve FIG. 3 in lieu of the dispensing tip FIG. 4-1 but such a configuration is less preferred for cost reasons and because it can limit the travel of the dispensing tip into the nostril.

The dispensing tip shown in cross section on FIG. 4-1 is inserted onto lightguide tip 1-2 and calibrated for output in radiometer port 1-4 and the resultant irradiance is displayed to the operator on display 1-5. The operator sets the timer 1-13 according to the displayed irradiance and inserts the dispensing tip into the quartz sleeve in the nostril to be treated.

The reflective element in the dispensing tip may be conical as shown in FIG. 4-1 and FIG. 4-2, or flat or downwardly domed as shown in FIG. 4-3. The selection of the shape of the reflective element depends upon the numerical aperture of the lightguide 1-3. For a lightguide with a NA of 0.5 the preferable shape of the reflector is a cone with a 30 degree taper. Testing this configuration gave a maximum irradiance ratio along the surface of the anterior nares of 1.7 as shown in FIG. 12. If the minimum irradiance delivered to any given area in the anterior nares is 1 minimum bactericidal dose which is approximately equal to 1 MED, then the maximum phototoxic reaction in the nares will be 1.7 MED which is a very mild redness harmless to the patient.

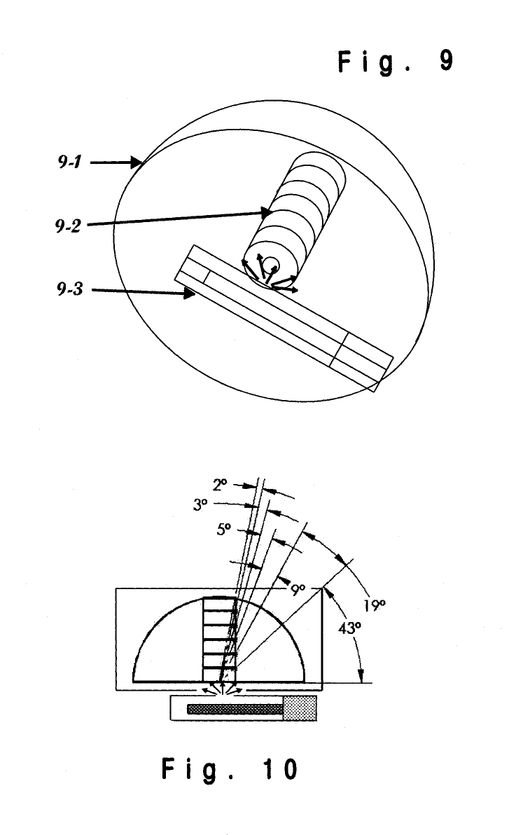

In FIG. 9 cylinder 9-2 represents one of the nares. The hemisphere 9-1 represents the lambertian radiation pattern of a low-pressure mercury lamp with its central aperture coincident with the center of the hemisphere and the proximal entrance of the cylinder. The energy density is homogeneous on the hemispherical surface. The low-pressure mercury lamp source is represented as 9-3. A two-dimensional cross section view is given in FIG. 10. The cylinder representing the nares is further subdivided into six sub-cylinders of equal size and surface area for illustrative convenience. As can be deduced from FIG. 10, the most proximal sub-cylinder to the lamp source receives energy at an included angle of radiation from the source of approximately 43 degrees. The included angle of the sub-cylinder at 1 cm is 5 degrees and the most distal (top) sub-cylinder section is only 2 degrees. Since every included degree has the same amount of energy we can see that the distal part of the cylinder at 1 cm only receives 5/42 or approximately ⅛ of the radiation of the proximal part of the cylinder. In a cylinder of 1 inch the ratio gets worse and is 2/42 or 1/20. As is explained below, this uneven irradiance cannot give a MBD without compromising patient safety.

From the literature, we know that a 3-log kill (99.9%) of MRSA requires 15 mj/cm2 of UVC at 254 nm. Also from the literature we know that 15 mj/cm2 of UVC will typically induce a 1 MED (minimal erythema dose) phototoxic reaction which causes the skin to slightly redden. The literature also reports that at 5 MEDs severe phototoxic events such as edema and blistering start to occur. These phototoxic events can create opening for the MRSA to enter the bloodstream with severe consequences to the patient's health.

A dosing level of 3 MEDs will redden the skin, kill the MRSA on the surface but not induce the severe phototoxic events that can endanger the patient's health. Any dosing up to 3 MEDs would be considered safe from a phototoxic reaction standpoint. Since the lambertian emitters placed at the entrance of the nostril apply radiation at a 8:1 rate from the proximal to distal parts of a 1 cm cylinder, we can see that they cannot be used safely for nasal decolonization. If the 1 MED dose for killing bacteria is achieved at the distal part of the nares, the MED dose at the proximal portion of the nares would be 8 MEDs, which is nearly 3 times the safe dose.

Using the directed radiation from lightguide and reflective element of the dispensing tip of the device disclosed herein an irradiance ratio of 1.7 was achieved for a 10 mm depth into the nares. This irradiance ratio means that if one section of the cylinder received 1 MED the maximum MED that any other portion of the cylinder will be 1.7 MED or roughly half the safe UVC dose. One MED of UVC is approximately equivalent to the minimum bactericidal dose.

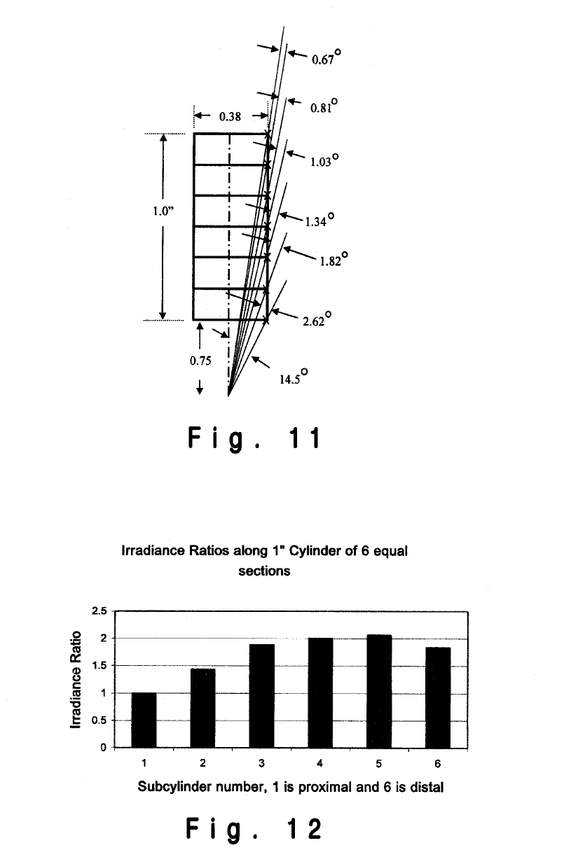

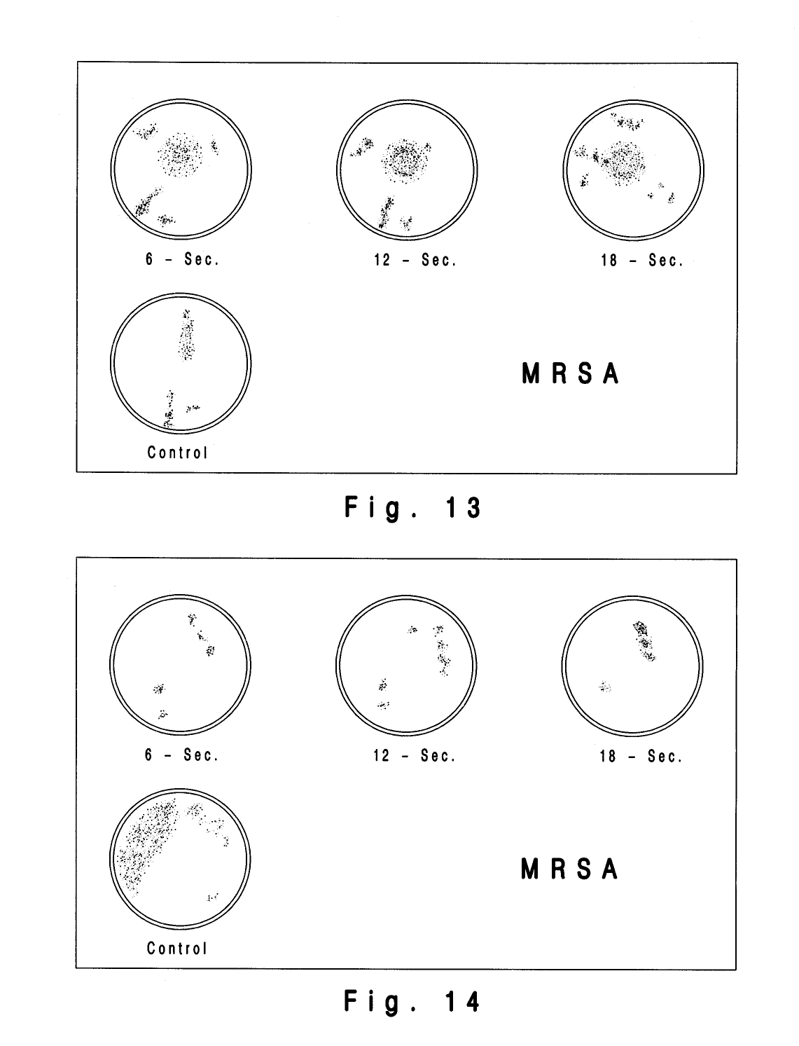

It is not at first obvious that the type of emission (lambertian, coherent, point source, etc.) from the light source affects the ability to effectively and safely apply the UVC radiation inside the nasal cavity. Inexpensive UVC sources such as low pressure mercury lamps cannot be used since the output cannot efficiently be put into a lightguide and shaped by optics or the lightguide or optical elements in the tip area. The delivery beam must be directed and shaped in order to keep the homogenity within the nasal cavity in the safe dosing range. Using a lightguide to convey the UVC from the lamp accomplishes much of the beam shaping required. The beam shaping ability of a 29 degree NA quartz lightguide is illustrated in FIG. 11. In this configuration the end of the lightguide is ¾″ from the entrance of the cylinder representing the nares. The included angle of radiation for the top segment of the 1 cm long cylinder is approximately half of the lower cylinder segment giving a 2:1 ratio of energy deposition. By adding the reflector at the end of the tip as shown in FIG. 4-1 we increase the radiation deposition and reduce the irradiance ratio to 1.7.

As was pointed out in the background information above a low-pressure mercury arc placed at the entrance of the anterior nares will deliver a minimum of 4 MED to the proximal surface if sufficient radiation is provided to kill MRSA at a 1 cm distance from the opening. Any dose above 3 MED poses significant health risk to the patient because the phototoxic reaction can open a route for the MRSA to enter the bloodstream.

It is another object of the invention to provide a device that minimizes unwanted or accidental skin and eye UVC irradiation. An epithelial layer of cells easily harmed by UVC covers the cornea of the eye. The lightguide and dispensing tip can be inserted into the sleeved nostril without UVC being emitted from the end since the shutter controls the emission from the lightguide. The insertion of the dispensing tip into the sleeve in the nasal cavity also provides for a centering of the projected beam within the nasal cavity to ensure that projected beam is not biased toward one side of the nasal cavity. The provision for emission-only when the dispensing tip is inside the cavity protects the operator, the treatment subject and other people in the vicinity of the device. With additional eye safety in mind, a very bright visible light is mixed with the invisible UVC radiation to warn people that the instrument is emitting UVC. One of the potential safety problems associated with low-pressure mercury and monochromatic UVC LED sources of prior art devices is that device output is mostly or completely invisible. Inadvertent eye or skin exposure can occur without either the operator or treatment subject being aware that irradiation of the skin or eye has occurred. This is particularly true if the device has no shutter or visible or audible warning of UVC emission from the device. The MRSA nasal decolonization device described herein is designed to emit UVC only when the dispensing tip is inside the nostril, and thus will not be emitting radiation when the device is warming up, or when the lightguide is being moved toward or away from the face. In non-medical settings it is desirable to have an obvious warning system of light emission since non-medical personnel in the community setting may not be as aware of the dangers of UVC as trained medical personnel. In a one embodiment of the device, the device sounds an audible warning whenever emissions from the lightguide are occurring.

The visible light used for warning is obtained from the same source as the UVC, the arc of the high-pressure mercury or xenon short-arc lamp. One of the strong emission lines in the spectrum of the mercury short-arc lamp occurs at 546 nm, which is close to the maximum sensitivity of the human eye to visible radiation.

The visible warning light also provides feedback to the user when the device is used to provide germicidal radiation to skin areas prior to surgical incision. The visible light and UVC light are intermixed and this visual feedback allows the operator to verify the geography and boundaries of the surface that is being decontaminated by the UVC.

An additional benefit of the green 546 nm light mixed with the UVC is that it is antimicrobial without having phototoxic effects. Another visible wavelength that can be selected is a blue wavelength at 405 nm, which is a strong emission peak of a short-arc mercury lamp. In a recent publication, radiation at a 405 nm wavelength was shown to be effective in killing MRSA. In that report, a 405 nm radiation fluence of 10 J/cm2 was demonstrated to have a 50% kill of MRSA in-vitro. It will be evident to those skilled in the art that various blue and green wavelengths could be combined to give effective antimicrobial activity and simultaneously provide a warning and aiming beam.

It is another object of the invention to provide a quartz nasal sleeve and dispensing tip FIG. 3 that allows the passage of UVC and visible light and also provides protection against potential cross-contamination from patient to patient. The quartz sleeve can be sterilized easily or disposed of after use. The sleeve and dispensing tip act as self-centering devices for the beam along the central axis of the nasal cavity and prevent aiming of the beam to one aspect of the nares which could cause a burn. FIG. 4-2 shows a tip that uses an extra band of tube material as an attenuator to further shape the output homogeneity of the beam. The preferred material for the dispensing tip is a tube of FEP (fluorinated ethylene propylene) fluoropolymer, but other UVC transmitting fluoropolymer compounds such as polychlortrifluroethylene (PCTFE) can be used. The preferred material for the nasal sleeve is quartz. Test data of FEP tubing with a nominal wall thickness of 0.007 inches showed about 50% UVC transmission losses due to the FEP. The UVC losses in the tip are compensated for during the calibration procedure since the tip is on the lightguide during calibration.

Since there are horizontal surfaces on the anterior portion of the nasal cavity known as the nasal vestibule shown in FIG. 7-1 and FIG. 7-2, radiation being applied to cover this area must be directed backward from the dispensing tip. This is accomplished by a reflective element, preferably cone shaped or dome shaped that is mounted in the most distal portion of the dispensing tip tube. This reflecting element also blocks radiation from entering the posterior nares and sinus areas, which are vulnerable to UVC radiation, because these areas do not have a cornified skin covering.

The reflective element shown on the distal tip of the dispensing tip FIG. 4-1 may be aluminum which is highly reflective in the UVC range, or it can be a plastic part coated with a UVC reflective paint incorporating barium sulfate or other compounds know to reflect well in the UVC. The reflective element can be held in place by using heat shrinkable FEP or by adhesive.

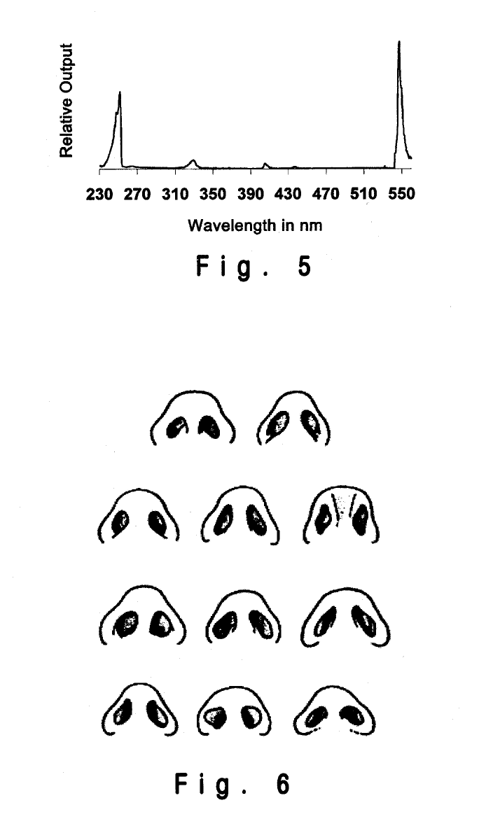

Another object of the invention is to provide anti-fungal radiation treatment for a condition of the human nails called onychomycosis. Fingernails and toenails can be infected with fungus that is located beneath the nail plate. Nail Fungus affects an estimated 2 percent to 18 percent of all people worldwide. Nail plates are formed of a tough protein called keratin, which is not easily penetrated by UVC. Since there are no proliferating cells within the nail plate the concerns of DNA damage to proliferating cells or phototoxic reaction in the nail is remote. In a preferred embodiment of the design, the UVC is mixed with green light at 546 nm as shown in the spectrum of FIG. 5. Green light has been demonstrated to have significant antifungal activity on Trichophyton rubrum, which is a common form of the fungus causing onychomycosis. UVC has also been shown to have significant antifungal properties for dermatophytic and saprophytic fungi, molds, yeasts, and bacteria that can play a role in nail infections.

For use on nail beds or for sterilization of surgical skin sites, the device can use the lightguide without the nasal dispensing tip to apply the radiation.

In one embodiment of the device using mercury short-arc lamp and a 3 mm lightguide it was possible to achieve irradiances at the tip of the lightguide exceeding 1.0 W/cm2 of UVC and about 2.8 W/cm2 of green light at 546 nm. Light transmission through a nail plate depends upon many factors including thickness, but reports in the literature show the nail plate passes approximately 0.01% of UVC and 10%-20% of visible light. The area under a 3 mm lightguide may be treated in 20 seconds delivering a fluence of about 2 mj/cm2 UVC and about 8.4 J/cm2 of green light at 546 nm to the nail bed. Each of these wavelengths has been reported to have significant photo-onycholytic effects on nail fungus at these fluences.

UVC light source 10 may be comprised of a high-pressure mercury short-arc lamp or xenon short-arc lamp with an ellipsoidal reflector as depicted in FIG. 2-1. In a preferred embodiment the lamp and reflector would be an integral unit to reduce the time required to replace and align the lamp. In another preferred embodiment the quartz tubing of the mercury short-arc lamp would be non-ozone producing. This eliminates the need for filtering out the 185 nm line in the mercury spectrum. The reflector can be a dichroic-coated glass reflector that reflects UVC and visible light, but passes infrared (IR) radiation. This eliminates the need for the optical filter assembly 11 to eliminate the IR emitted by the lamp. Such integral-reflector high-pressure short-arc lamps for mercury and xenon with dichroic coatings that reflect UVC and visible but pass IR are commercially available (Philips, Eindhoven, Netherlands). Alternatively, the UVC source 10 can be a pulsed xenon lamp or xenon flashlamp as illustrated as FIG. 2-2. Xenon flashlamps can produce as much as 15% of their output as UVC, and can be coupled into lightguides. In a preferred embodiment, the xenon flashlamp incorporates an internal reflector producing a collimated output beam, which can be efficiently focused by a fused silica lens as shown. The focal length of the lens can be selected to either maximize UVC collection or to minimize the irradiance ratio of the projected beam depending upon the NA of the lightguide that is selected. The optical filter 11 may be placed between the flashlamp and lens, or placed between the lens and output port if the dichroic coating of filter 11 has sufficient bandwidth for rejection of off-axis radiation. In another embodiment UVC source 10 is an excimer laser, forward emitting excimer lamp, or excimer photon amplifier as illustrated in FIG. 2-3. The xenon iodideexcimer dimer produces 253 nm radiation, and excimer species such as KrF at 248 nm and Cl2 at 259 nm are also good choices. Alternatively, a frequency-quadrupled Nd:Yag laser that operates at 266 nm may be used as the UVC source. The disadvantage of excimer or other laser sources is the high cost of these lasers and lamp sources and the need to add a separate beam source for the visible beam. The addition of a visible light beam to an ultraviolet beam is accomplished by a folding mirror and is well understood by those skilled in the art of photonics.

The germicidal efficacy of the device was tested as described in Example 1 and Example 2 below.

### Example 1

An embodiment of the device was constructed using a 100 watt mercury short-arc lamp as depicted in FIG. 2-1 as the UVC light source and fused silica dichroic filters were used for optical filter 11. Lightguide 3 was a UVC liquid lightguide with a 3 mm diameter core and 1 meter length. The dispensing tip in FIG. 4 was constructed of 0.007-inch wall FEP tubing without reflective element to allow projection of the beam. The UVC output of the tip was measured at 60 mw by a calibrated external radiometer (Molectron Model 150-50c) through a green-light blocking fused silica dichroic filter. The output spectrum of the lightguide tip is shown in FIG. 5.

Individual aqueous suspensions of actively growing, fresh cultures of Staphylococcus aureus, MRSA and Beta Hemolytic Streptococcus Group A were prepared to a concentration of approximately 1.4 million colony forming units/ml. Using sterile swabs, trypticase soy 5% sheep blood agar plates were streaked with the individual suspension in order to achieve a confluent “lawn” of bacterial growth. Each of these inoculated plates, with the Petri dish lid removed, was exposed to the timed beam of UV light. A control plate inoculated with each organism has no UV beam exposure. All plates were incubated for 18-24 hours at 35 degrees C. in an atmosphere of 5% carbon dioxide.

As clearly depicted in the photograph for MRSA no bacterial growth occurred in the circular area of UV beam exposure. There was complete bacterial killing and remarkably no visible damage or hemolysis of the sensitive blood agar media. The control plates not exposed to the UV beam had complete confluent growth of each organism.

### Example 2

The device as described in Example 1 with the output shown in FIG. 5 was used as the irradiation source. Individual 12×75 mm sterile test tubes were prepared, each containing 50 ul of an aqueous suspension with approximately 70,000 colony forming units of fresh, actively growing Staphylococcus aureus, MRSA and Beta Hemolytic Streptococcus Group A. Each tube was exposed to the timed UV light beam. Control tubes had no UV exposure.

After the exposure, 10 ul from each tube was transferred to individual trypticase soy 5% sheep blood agar plates, streaked for isolation and incubated for 18-24 hours at 35 degrees C. in an atmosphere of 5% carbon dioxide.

As depicted in the photograph for the MRSA test there was a virtual 100% kill of all organisms exposed to the UV beam, whereas the control tube organisms were completely viable.

The antifungal efficacy of the device for use on human nails was tested in vivo and the results on one patient showed complete visual clearing of all the signs of the fungus in 3 treatments.

While the present invention or inventions have been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

1. A device for decolonizing microbes on the skin, under nail beds, and in body cavities comprising:
a) a base unit with a UVC light source, optical collection and filtering means, an output port; and
b) a lightguide connected to the base unit,
c) means for calibration of the UVC output of the lightguide, and
d) a dispensing tip on the lightguide with provisional accessories.

2. The device of claim 1 where the UVC light source is taken from the group comprised of a high pressure, short-arc mercury or xenon lamp, a xenon flashlamp, an excimer laser, or a UVC laser.

3. The device of claim 1 wherein the dispensing tip is configured for insertion into the nasal cavity and incorporates a reflective element that directs radiant energy backwards and sideways onto the sides of the anterior nares.

4. The device of claim 1 wherein the dispensing tip can be removed for direct irradiation from the lightguide onto nail beds or surgical sites.

5. The device of claim 3 for use in decolonization of nasal MRSA wherein the dispensing tip incorporates a provisional accessory comprising a separate hollow UVC transmissive sleeve adapted for insertion into the nasal cavity prior to the insertion of the dispensing tip.

6. The device of claim 1 where the UVC output is in the range of 230 nm-280 nm and a visible wavelength or combination of visible wavelengths between 400 nm and 700 nm is included in the radiant output.

7. The device of claim 1 where the UVC output is in the range of 230 nm-280 nm and the visible wavelength is largely comprised of green light with a peak at 546 nm.

8. The device of claim 4 where the total output of the UVC from the dispensing tip is greater than 35 mw and the visible light output is greater than 200 mw.

9. The device of claim 1 where the UVC output is in the range of 230 nm-280 nm and the visible wavelength is largely comprised of light at 405 nm and 436 nm.

10. The device of claim 1 where the optical filtering means is comprised of dichroic filters on a fused silica or quartz substrate.

11. The device of claim 1 where the lightguide is comprised of a UVC transmitting liquid medium or of fused silica or quartz.

12. The device of claim 1 where the UVC transmissive dispensing tip is taken from the group comprised of fluorinated ethylene propylene (FEP) or other fluoropolymer and a reflective element taken from the group of an aluminum or a UVC reflective paint is mounted to the distal portion of the tip.

13. The device of claim 1 where the UVC transmissive dispensing tip is taken from the group comprised of quartz, fused silica or sapphire and a reflective element is taken from the group comprised of aluminum or a UVC reflective paint mounted to the distal portion of the tip.

14. The device of claim 1 where the body cavity treated is the human nose and the microbe being decolonized is Methicillin-resistant Staphylococcus aureus (MRSA).

15. The device of claim 1 where the transmissive dispensing tip has provisions for UVC opaqueness, UVC attenuation, or UVC reflective masking to control the radiation pattern emitted from the tip.

16. The device of claim 1 where the calibration means is integral to the base unit, and the calibration information can be displayed to the operator.

17. A method of decolonizing MRSA from the nasal cavity by irradiating the anterior nares with UVC light within the wavelength range of 230 nm to 280 nm wherein the radiation is delivered by a lightguide to a dispensing tip that incorporates a reflective element and the nostril is expanded in advance of the irradiation by a UVC transmissive cylindrical sleeve.

18. A method of decolonizing MRSA from the nasal cavity by irradiating the anterior nares with combination UVC light within the wavelength range of 230 nm to 280 nm and antimicrobial visible light within the range of 400 nm to 700 nm wherein the radiation is delivered by a lightguide to a dispensing tip that incorporates a reflective element and the nostril is expanded in advance of the irradiation by a UVC transmissive cylindrical sleeve.
